(19) United States
(12) Patent Application Publication
(10) Pub. No.: US 2014/0228869 A1
(43) Pub. Date: Aug. 14, 2014

(54) THROMBECTOMY CATHETER

(71) Applicant: MEDRAD, INC., Indianola, PA (US)

(72) Inventors: Michael J. Bonnette, Minneapolis, MN (US); Eric J. Thor, Arden Hills, MN (US)

(73) Assignee: Medrad, Inc., Indianola, PA (US)

(21) Appl. No.: 13/766,367

(22) Filed: Feb. 13, 2013

(51) Int. Cl. A61B 17/3203

(52) U.S. Cl. CPC A61B 17/32037
USPC 606/159

(57) ABSTRACT

A thrombectomy catheter includes a catheter body extending from a catheter proximal portion to a catheter distal portion and including a catheter intermediate portion, the catheter body includes an aspiration lumen and an infusion lumen extending along the catheter body, wherein the aspiration lumen includes an aspiration orifice open at a distal end of the catheter body.

### FIELD

Medical devices, and more specifically to thrombectomy catheters and procedures.

### BACKGROUND

A thrombectomy is a medical procedure used to remove a blood clot (thrombus) from a vessel, such as an artery or vein. If a thrombus is not removed, it may obstruct blood flow. One technique to perform a thrombectomy is to use a catheter having an infusion lumen, used to break up the thrombus, and an aspiration lumen, used to vacuum up the thrombus and emboli.

In some examples, thrombectomy procedures are conducted with complex catheter systems configured to provide multiple jets of high pressure fluid, such as saline supplied at pressures of 10,000 psi or more. Supplying high pressure fluid correspondingly requires a high pressure pump. Pumps for a high pressure thrombectomy procedure may have limited utility for other medical procedures (e.g., medication and contrast infusion and the like).

Additionally, the thrombectomy catheters used in these procedures are constructed with complex manifolds, fluid jet exhaust features and the like to distribute jets of fluid for the removal of thrombus from a vessel. Furthermore, these catheters are constructed with robust materials to permit the delivery and distribution of high pressure fluids. These thrombectomy systems are correspondingly expensive, require multi-step manufacturing techniques and further require specialized equipment for operation (for instance a high pressure pump, as described above).

### OVERVIEW

One example of the present disclosure can include a catheter body extending from a catheter proximal portion to a catheter distal portion and including a catheter intermediate portion, the catheter body includes an aspiration lumen and an infusion lumen extending along the catheter body, wherein the aspiration lumen includes an aspiration orifice open at a distal end of the catheter body.

In another example of the present disclosure, the catheter body includes an integral homogenous cross-section profile and includes a multi-durometer hardness varying along the catheter body's length such that the catheter proximal portion has a relatively high durometer and the catheter distal portion has a relatively low durometer, with respect to each other.

In still another example of the present disclosure, the infusion lumen extends along the catheter body towards the distal portion and includes a single infusion orifice that is configured to direct a fluid jet radially away from a longitudinal axis of the catheter body.

In yet another example of the present disclosure, the distal end of the catheter body includes an aspiration orifice distal member including a proximal portion extending from the distal end of the catheter body having an opening sized similar to the aspiration lumen and a distal portion having an opening wider than the aspiration lumen.

A particular example discloses a thrombectomy catheter comprising a catheter body extending from a catheter proximal portion to a catheter distal portion; an aspiration lumen extending through the catheter body from the catheter proximal portion toward the catheter distal portion, the aspiration lumen including an aspiration orifice near the catheter distal portion, wherein the distal end of the catheter body includes an aspiration orifice distal member including a proximal portion extending from the distal end of the catheter body having an opening sized similar to the aspiration lumen and a distal portion having an opening wider than the aspiration lumen; and an infusion lumen extending along the catheter body towards the distal portion and having a single infusion orifice located in a side wall of the catheter body that is configured to direct a fluid jet radially away from a longitudinal axis of the catheter body.

Another particular example discloses a thrombectomy catheter comprising a catheter body extending from a catheter proximal portion to a catheter distal portion and including a catheter intermediate portion, the catheter body including an aspiration lumen and an infusion lumen extending along the catheter body, the catheter body having an integral homogenous cross-section profile and having a multi-durometer hardness varying along the catheter body's length such that the catheter proximal portion has a relatively high durometer value and the catheter distal portion has a relatively low durometer value, with respect to each other; wherein the aspiration lumen includes an aspiration orifice open at a distal end of the catheter body; and wherein the infusion lumen extends along the catheter body towards the distal portion and includes a single infusion orifice that is configured to direct a fluid jet radially away from a longitudinal axis of the catheter body.

Another particular example discloses a thrombectomy catheter comprising a catheter body extending from a catheter proximal portion to a catheter distal portion and including a catheter intermediate portion, wherein the catheter proximal portion has a relatively high durometer value and the catheter distal portion has a relatively low durometer value, with respect to each other; the catheter body including an aspiration lumen extending through the catheter body from the catheter proximal portion toward the catheter distal portion, the aspiration lumen including an aspiration orifice open at a distal end of the catheter body, wherein the aspirating orifice is free from structural obstructions at the distal end of the catheter body and wherein the distal end of the catheter body includes an aspiration orifice distal member including a proximal portion extending from the distal end of the catheter body having an opening sized similar to the aspiration lumen and a distal portion having an opening wider than the aspiration lumen; the catheter body further including an infusion lumen extending along the catheter body towards the distal portion with an infusion orifice extending through the catheter body to direct a fluid jet away from the catheter body.

Another particular example discloses a thrombectomy catheter comprising a catheter body extending from a catheter proximal portion to a catheter distal portion and including a catheter intermediate portion, the catheter body including an aspiration lumen and an infusion lumen extending along the catheter body, the catheter body having an integral homogenous cross-section profile and having a multi-durometer hardness varying along the catheter body's length such that the catheter proximal portion has a relatively high durometer value and the catheter distal portion has a relatively low durometer value, with respect to each other; wherein the aspiration lumen extends through the catheter body from the catheter proximal portion toward the catheter distal portion, the aspiration lumen including an aspiration orifice open at a distal end of the catheter body, wherein the distal end of the catheter body includes an aspiration orifice distal member including a proximal portion extending from the distal end of the catheter body a distal portion, wherein the distal portion has a greater cross-sectional area than the cross-sectional area of the proximal portion; and wherein the infusion lumen extends along the catheter body towards the distal portion with an infusion orifice extending through the catheter body to direct a fluid jet away from the catheter body.

Another particular example discloses a thrombectomy catheter comprising a catheter body extending from a catheter proximal portion to a catheter distal portion and including a catheter intermediate portion, the catheter body including an aspiration lumen and an infusion lumen extending along the catheter body, the catheter body having an integral homogenous cross-section profile and having a multi-durometer hardness varying along the catheter body's length such that the catheter proximal portion has a relatively high durometer value and the catheter distal portion has a relatively low durometer value, with respect to each other; wherein the aspiration lumen extends through the catheter body from the catheter proximal portion toward the catheter distal portion, the aspiration lumen including an aspiration orifice open at a distal end of the catheter body; and wherein the infusion lumen extends along the catheter body towards the distal portion with an infusion orifice extending through the catheter body to direct a fluid jet away from the catheter body.

Another particular example discloses a thrombectomy catheter comprising a catheter body including an aspiration lumen extending though the catheter body and open at an aspiration orifice; an infusion body including a fluid delivery lumen extending to an infusion orifice, the infusion body extending through the aspiration lumen; and an expanded member coupled to a distal end of the infusion body and located distally from the infusion orifice.

Another particular example discloses a thrombectomy system comprising a fluid delivery device; an aspirator; and a thrombectomy catheter with a first port coupled to the fluid delivery device and a second port coupled to the aspirator, wherein the thrombectomy catheter includes: a catheter body extending from a catheter proximal portion to a catheter distal portion and including a catheter intermediate portion, wherein the catheter proximal portion has a relatively high durometer value and the catheter distal portion has a relatively low durometer value, with respect to other; an aspiration lumen extending through the catheter body from the catheter proximal portion toward the catheter distal portion, the aspiration lumen including an aspiration orifice open at a distal end of the catheter body, wherein the distal end of the catheter body includes an aspiration orifice distal member including a proximal portion extending from the distal end of the catheter body having an opening sized similar to the aspiration lumen and a distal portion having an opening wider than the aspiration lumen; the catheter body further including an infusion lumen extending along the catheter body towards the distal portion with an infusion orifice extending through the catheter body to direct a fluid jet away from the catheter body.

Another particular example discloses the thrombectomy system of the previous paragraph wherein the aspirator includes a vacuum source including a plurality of syringes ganged together via a stop cock style manifold.

Another particular example discloses the thrombectomy catheter of any of the previous paragraphs wherein the single infusion orifice is recessed proximally away from the aspiration orifice.

Another particular example discloses the thrombectomy catheter of any of the previous paragraphs wherein the distal end of the catheter body includes an aspiration orifice distal member including a proximal portion extending from the distal end of the catheter body having an opening sized similar to the aspiration lumen and a distal portion having an opening wider than the aspiration lumen.

Still another particular example discloses an injector system comprising a housing holding a high pressure pump, a low pressure pump, and an aspiration module; wherein a thrombectomy catheter is configured for coupling to the high pressure pump or the low pressure pump; the high pressure pump further comprising a single piston pump capable of delivering fluid at pressures of between 5000 psi to 10,000 psi; and the low pressure pump further comprising a multi-piston pump capable of delivering fluids at between 500 psi to 1500 psi.

Another particular example discloses the injector system of the previous paragraph wherein the high pressure pump and the low pressure pump are configured to operate independently of each other.

These examples can be combined in any permutation or combination. This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows a thrombectomy catheter, in accordance with one embodiment of the present disclosure.

FIG. 2 shows a cross-section of the thrombectomy catheter of FIG. 1.

FIG. 3 shows a perspective view of the distal portion of the thrombectomy catheter of FIG. 1.

FIG. 4 shows side view of the distal portion of the thrombectomy catheter of FIG. 1.

FIG. 5 shows an end view of an aspiration orifice member.

FIG. 6A shows a side view of the thrombectomy catheter in a vessel with thrombus lodged in a wide mouth distal member with the thrombus annularly engaged by the wide mouth perimeter.

FIG. 6B shows a side view of the thrombectomy catheter of FIG. 6A with the thrombus collapsed within the wide mouth distal member and translated toward the proximal catheter end.

FIG. 6C shows a side view of the thrombectomy catheter of FIG. 6B with the thrombus collapsed into the smaller diameter portion of the catheter and translated toward the proximal catheter end.

FIG. 7 shows a cross-section of a thrombectomy catheter, in accordance with one embodiment of the present disclosure.

FIG. 8 shows a distal end of a thrombectomy catheter, in accordance with one embodiment of the present disclosure.

FIG. 9A shows a portion of a thrombectomy system, in accordance with one embodiment of the present disclosure.

FIG. 9B shows an injector system, in accordance with one embodiment of the present disclosure.

FIG. 10 shows a perspective view of a vacuum source, in accordance with one embodiment of the present disclosure.

FIG. 11 shows a front view of the vacuum source of FIG. 10.

FIG. 12A shows a schematic view of one example of an injector system, according to one embodiment of the present disclosure.

FIG. 12B shows a schematic view of one example of an injector system, according to one embodiment of the present disclosure.

FIG. 13A shows a side view of a thrombectomy catheter according to one embodiment of the present disclosure.

FIG. 13B shows a detailed cross sectional view of an expanded member for used with the thrombectomy catheter shown in FIG. 13A.

FIG. 14 shows a detailed view of the thrombectomy catheter of FIG. 13A with a plug at an aspiration orifice.

FIG. 15 shows another detailed side view of the thrombectomy catheter of FIG. 14 with the plug mechanically driven into an aspiration lumen.

### DETAILED DESCRIPTION

FIG. 1 shows a side view of a thrombectomy catheter 100 in accordance with one embodiment of the present disclosure. As will be described in detail below, the thrombectomy catheter 100 is configured to provide a pressurized fluid at a distal end for the removal of thrombus from a vessel. Additionally, the thrombectomy catheter is configured to provide a vacuum source (aspiration) at the catheter distal end for removal of thrombus removed with the pressurized fluid. The thrombectomy catheter 100 generally includes a catheter body 102 extending from a catheter proximal portion 104 to a catheter distal portion 108. A catheter intermediate portion 106 extends between catheter proximal and distal portions 104, 108. The catheter body 102 is configured, in one example to provide a catheter distal portion 108 more flexible than the catheter proximal portion 104 to facilitate the navigation of the catheter body 102 through vasculature of the subject. The catheter body 102 includes an aspiration lumen 110 and an infusion lumen 111 extending along the catheter body 102 from the catheter proximal portion 104 toward the catheter distal portion 108.

Referring to FIG. 1, the infusion lumen 111 is coupled to a side port 122 that can be coupled to a fluid delivery device, as will be discussed below. The infusion lumen 111 is configured to deliver fluid under pressure to the catheter distal portion 108, for example, to a jet orifice used in a thrombectomy procedure. The jet orifice provides a jet of the fluid at pressures of around 1500 psi for hydrodynamic engagement with thrombus although other pressures may be obtained. The aspiration lumen 110 is coupled to a central port 120 that can be coupled to a vacuum apparatus.

In use, the thrombectomy catheter 100 is inserted into a vessel, such as a vein or artery, and fluid is delivered to the catheter distal portion 108 via the infusion lumen 111. The fluid is delivered through one or more jets, and hydrodynamically breaks up thrombus within the vessel (e.g., through concentrated fluid pressure, fluid velocity, and fluid flow volume). For instance, the fluid impacts the thrombus and mechanically macerates the thrombus through this engagement. As discussed below, the aspiration lumen 110 receives the broken up thrombus, through a widened aspiration orifice distal member 114, and delivers it through port 120 to a waste unit such as a collection bag, vial, chute and the like.

### Catheter Body

In one embodiment, the catheter body 120 is formed such that the distal portion 108 is relatively flexible, and the proximal portion 104 is stiff relative to the distal portion 108. Relative flexibility of the distal portion 108 allows the catheter body 120 to flexibly follow or navigate the vessel for ease of insertion. The stiffer proximal portion 104 of the catheter body 120 allows for more torqueability and easier advancement along a guide wire, for example. In one example, approximately the distal 6 inches of the catheter body 120 has a lower durometer hardness than the rest of the catheter body. One exemplary catheter uses 6533 PEBAX for the distal portion 108 and 7233 PEBAX for the proximal portion 104, with the 7233 PEBAX having a lower durometer value than the 6533 PEBAX.

In another example, the catheter proximal portion 104 has a high durometer value, the catheter intermediate portion 106 has a relatively medium durometer value, and the catheter distal portion 108 has a relatively low durometer value, with respect to each of the other of the proximal, intermediate, and distal catheter portions 104, 106, 108 of the catheter body. As with the previous example, the lower durometer value catheter distal portion 108 and intermediate portion 106 facilitate the delivery and navigation of the catheter within the vasculature. For instance, the catheter body 102 is navigable through tortuous vasculature. The relatively higher durometer value of the catheter distal portion (and to a lesser extent the intermediate portion) assists in providing pushability and torqueability to the catheter body 102.

For example, where the catheter body 102 includes three or more durometer values, as described above, the catheter body 102 is formed of polyurethane or PEBAX with the catheter proximal portion 104 having a durometer DP of Shore hardness A-A2, the catheter intermediate portion 106 having a durometer DI of B1-B2, and the catheter distal portion 108 having a durometer DD of C1-C2, where DP>DI>DD. Stated another way, the catheter body 102 has a gradually decreasing durometer value (and corresponding stiffness) from the catheter proximal portion 104 to the catheter distal portion 108.

Optionally, the catheter body 102 with the multi-durometer value construction is formed by a co-extrusion process. In one example, a Total Intermittent Extrusion (TIE) process is used. In a TIE process two or more different durometer value polymer resins are extruded from separate dies in line, with the higher durometer value polymer used for the proximal end of the catheter body (e.g., the catheter proximal portion 104) and the lower durometer value polymer used for the distal end of the catheter body (e.g., the catheter distal portion 108), with an intermediate transition zone therebetween, such as the intermediate portion 106 of the catheter body 102. As discussed previously, in one example a 6233 PEBAX is used for the distal end and a 7233 PEBAX is used for the proximal end. In one example, the extruded catheter profile is homogenous along the length of the catheter with the durometer value of the catheter varying along the length. That is to say, the catheter materials are gradually mixed in various amounts according to the desired durometer value and thereafter extruded. In another example, varying of the catheter body 102 durometer value includes extruding one of the proximal and distal portions 104, 108 (e.g., the materials having one of the higher or lower durometer values, respectively) in an end to end fashion and then switching the extrusion resin to a lower or higher durometer material, for the distal and proximal portions 108, 104, respectively.

In other examples, the catheter profile can include two or more layers of material. For example, in one embodiment, the transition zone between the distal end and the proximal end can include a mix of material as the durometer values change from the 6233 PEBAX to the 7233 PEBAX. Stated another way, multiple layers of differing durometer materials are coextruded and alternatively interrupted or added to provide the desired durometer value for the overall catheter body 102.

In still other examples, the catheter body 102 is formed with other processes as known to those of skill in the art, including, but not limited to, shrinking tubing along a lumen liner, welding catheter tubes with varying diameter together at junctions and the like.

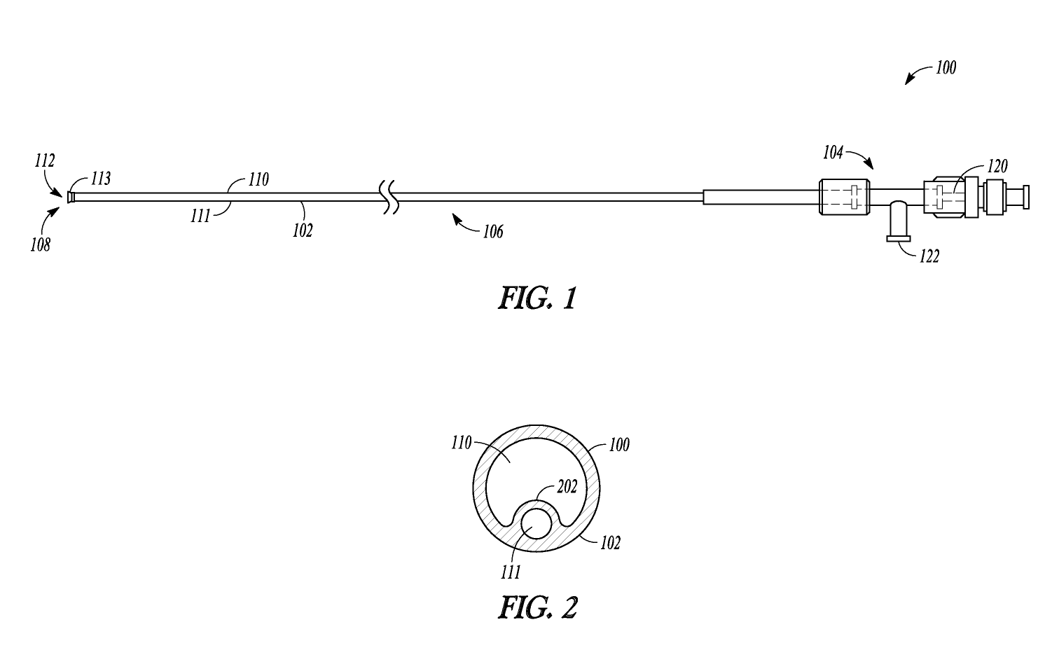

FIG. 2 shows a cross-section of the thrombectomy catheter 100, in accordance with one embodiment of the present disclosure. In this example, the infusion lumen 111 is located off-center relative to the aspiration lumen 110 with a septum 202 separating the infusion lumen 111 from the aspiration lumen 110. The catheter body 102 includes an exterior catheter surface and an interior catheter surface, and the aspiration lumen 110 is circumscribed by the interior catheter surface. As shown, exterior catheter surface is featureless and the infusion lumen 111 is recessed relative to the exterior catheter surface. The recessed infusion lumen 111 facilitates the delivery and navigation of the catheter body 102 by providing an isodiametric cylindrical profile, in one example. Further, the recessed infusion lumen 111 includes a partial profile within the aspiration lumen 110 formed by the infusion lumen sidewall. The infusion lumen 111 is positioned at the perimeter of the aspiration lumen 110 to ensure the largest overall profile is available for aspiration of thrombus particles through the aspiration lumen without interference by an infusion lumen, for instance an infusion lumen positioned centrally with the aspiration lumen or resting along an infusion lumen wall (as with a lumen infusion sidewall separate from an aspiration lumen sidewall).

In one embodiment, the catheter body 102 has a diameter of 6 French (Fr) and is inserted using a 0.014 inch guidewire. In another embodiment, the catheter body 102 has a diameter of 8 French and uses a 0.014 inch to a 0.035 inch guidewire for insertion. Optionally, the catheter body 102 includes other diameters and is accordingly usable with corresponding guidewires for delivery.

In one example, the catheter body 102 has a homogenous cross-sectional profile. In other words, the cross-section profile of the catheter body 102, including the infusion lumen 111 and the aspiration lumen, is formed simultaneously and is correspondingly without any sort of bond line or weld line between the sidewall of the infusion lumen 111 and the sidewall of the aspiration lumen 110. This contrasts to a structure where the two lumens are formed separately and then bonded together at a later stage. The homogenous cross-section of the catheter body 102 provides for a more robust structure that is resistant to fracture or peeling of one lumen relative to the other lumen since any bending or torquing of the catheter or the pressures within the lumens will not cause a rupture of a bond line between the two lumens. Alternatively, the aspiration and infusion lumens 110, 111 are separately formed and thereafter coupled together for instance, with welds, adhesives, reflowing and the like.

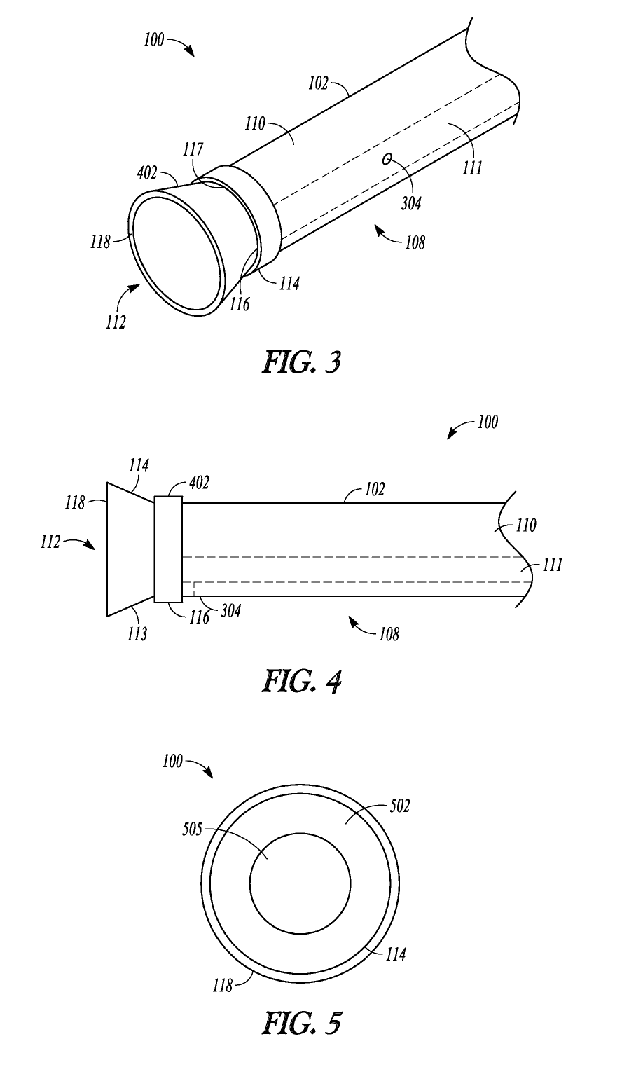

FIG. 3 shows a perspective view of the distal portion 108 of the thrombectomy catheter 100, in accordance with one embodiment. FIG. 4 shows a side view of the distal portion 108 of the thrombectomy catheter 100. As shown in each of these examples, the distal portion includes an aspiration orifice 112 and an infusion orifice 304. As described herein, the aspiration and infusion orifices 112, 304 cooperate during a thrombectomy procedure to hydrodynamically remove thrombus from a vessel, macerate the thrombus and aspirate the thrombus from the vessel.

### Infusion System

Referring again to FIGS. 3 and 4, the infusion lumen 111 extends along the catheter body 102 toward the distal portion 108 with the infusion orifice 304 extending through the catheter body (e.g., through a sidewall of the catheter body adjacent to the infusion lumen 111) to direct a fluid jet away from the catheter body 102. As described herein, the infusion lumen 111 is fluidly coupled with a fluid source configured to provide pressurized fluid, such as saline, for instance at a pressure of around 1500 psi or less. The pressurized fluid is delivered through the infusion orifice 304 and is metered by the orifice 304 to form the fluid jet for the thrombectomy procedure.

In one embodiment of the present disclosure, a single infusion orifice 304 is provided that is configured to direct a fluid jet radially away from a longitudinal axis of the catheter body 102. For instance, the single infusion orifice 304 is directed away from the catheter body 102 to ensure the fluid jet generated at the infusion orifice impinges upon thrombus in a vessel surrounding the catheter body 102. By rotating the catheter body 102 (for instance a catheter body including a higher durometer value proximal portion 104), the infusion orifice 304 and the corresponding fluid jet travel the full measure of the vessel and can thereby remove all thrombus around the catheter distal portion 108. In one example, the infusion orifice 304 has a diameter of about 0.009 inches. In another example the infusion orifice 304 has a diameter of about 0.012 inches. Optionally, the infusion orifice 304 has a diameter configured to generate a fluid jet having a desired velocity and fluid flow rate according to the source of pressurized fluid (e.g., the pressure and flow rate for a pump system coupled with the catheter body 102). Stated another way, the infusion orifice 304 shape and size are configured to cooperate with a fluid source to provide a fluid jet with desired velocity and flow rate values.

In the example described above, a single infusion orifice 304 is provided. In other examples, a plurality of infusion orifices 304 are provided at one or more locations on the catheter body 102 (e.g., radially around the catheter distal portion 104, longitudinally, and the like). A single infusion orifice 304, as shown in FIG. 4 concentrates the hydrodynamic energy of the infusion fluid to better break up the thrombosis. That is to say, by using a single infusion orifice 304, even a low pressure fluid source (for instance, 1500 psi or less having a low flow rate of 1 to 3 cc) is used to generate a fluid jet at the orifice 304 with sufficient hydrodynamic energy to perform a thrombectomy procedure normally reserved for fluid sources providing fluid at high pressure (e.g., 10,000 psi or more). The concentrated fluid jet at the infusion orifice 304 may then be traversed around the body vessel to provide similar efficacy to high pressure thrombectomy treatments using catheters that have a plurality of jet orifices and robust construction sufficient to deliver high pressure fluids.

Different embodiments of the thrombectomy catheter 100 use different infusion fluid flow rates. One example catheter uses a flow rate of about 1.5 cc/sec to provide a fluid jet at the infusion orifice 304 configured to remove and macerate thrombus. Another example uses about 2 cc/sec. Still another example uses about 3 cc/sec. As described above, the velocity of and flow rate of the infusion fluid leaving the infusion orifice 304 is dependent on the flow rate and pressure of the fluid source and the size and shape of the infusion orifice 304. As discussed herein below, a low pressure fluid source, such as a medication or contrast injector is used as the fluid source for the thrombectomy catheter 100. The thrombectomy catheter 100 described herein with the infusion orifice 304 and infusion lumen 111 thereby provides a thrombectomy system configured to effectively remove and macerate thrombus while using low pressure and low flow rate (e.g., medication and contrast) injectors and does not necessarily require high pressure fluid sources otherwise used with other thrombectomy procedures.

The infusion lumen 111 and the infusion orifice 304 are configured, in one example, to mitigate hemolysis, the destruction of blood cells through hydrodynamic energy. The present system constrains the infusion velocity within a range of from about 20 m/sec to about 30 m/sec to mitigate hemolysis. The infusion orifice 304, in one example, is sized and shaped to cooperate with the flow rate through the catheter (and accordingly cooperates with the pressurized fluid source) to ensure the infusion velocity at the orifice 304 is between around 20 m/sec to about 30 m/sec. By concentrating the infusion flow through the infusion orifice 304 having a specified diameter and shape and a single location on the catheter body 102, the infusion velocity is readily controllable while at the same time providing a localized jet of infusion fluid for maceration of thrombus.

### Aspiration System

In this example, the aspiration lumen 110 includes an aspiration orifice 112 that is open at a distal end 113 of the catheter body 102. A radiopaque collar 402 is in one example located on the distal portion 108. The radiopaque collar assists with imaging of the catheter distal portion 108 during insertion and navigation through a vessel, under fluoroscopic viewing.

In one example, the distal end 113 includes a widened aspiration orifice distal member 114 (e.g., a wide mouth portion providing a larger profile than an adjacent portion of the catheter body 102). The widened aspiration orifice distal member 114 includes a proximal portion 116 coupled to an end 117 of the catheter body 112 as shown in FIG. 3. The widened aspiration orifice distal member 114 includes an opening at the proximal portion 116 sized similar to the aspiration lumen 110. The distal end 118 of the widened aspiration orifice distal member 114 includes an opening wider than the aspiration lumen 110. The widened aspiration orifice distal member 114 is attached to the end of the catheter body 102 by one or more of heat bonding, welding, adhering, reflowing and the like. This widened, funnel-shaped, distal member 114 (e.g., a wide mouth feature) provides for improved aspiration, as will be further discussed below.

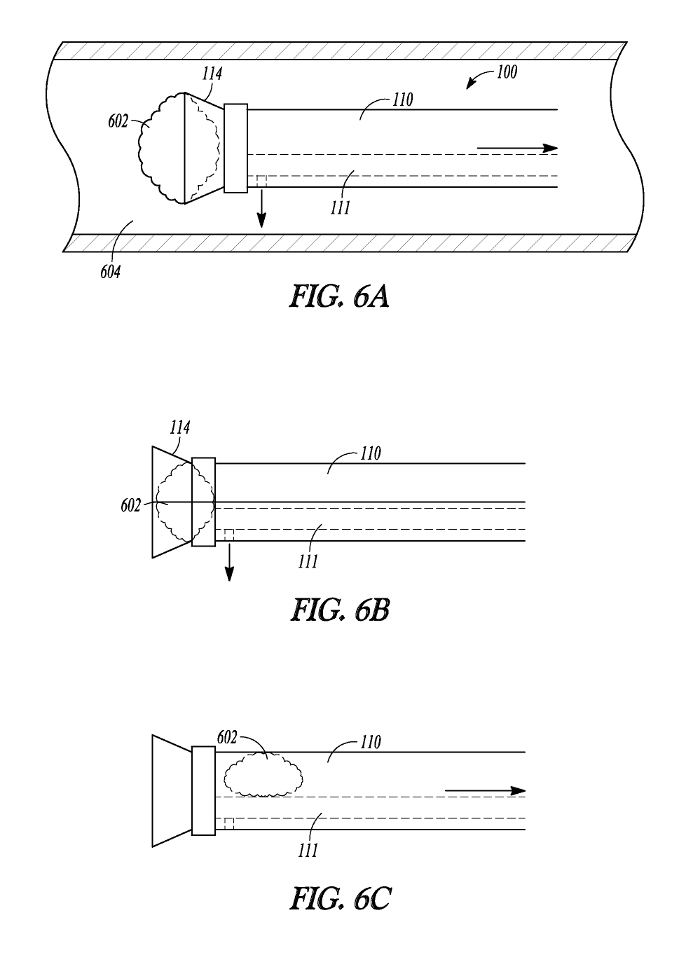

FIG. 5 shows an end view of the widened aspiration orifice distal member 114, unattached to the catheter body. FIG. 6A shows a side view of the thrombectomy catheter 100 in a vessel 604 with the widened aspiration orifice distal member 114 coupled with the catheter 100 and engaged with thrombus 602.

Referring again to FIG. 5, the wide mouth of widened aspiration orifice distal member 114 defines an inner sloping surface 502 that extends from the distal end 118 inward to a proximal portion of the widened aspiration orifice distal member 114 that is attached to the catheter body 104 and communicates with the aspiration lumen 110. In one example, the interface between the widened aspiration orifice distal member 114 and the aspiration lumen 110 is relatively smooth or flush to facilitate the transition of thrombus form the widened aspiration orifice distal member 114 to the aspiration lumen 110.

As shown in FIGS. 5 and 6A, the volume within the widened aspiration orifice distal member 114 from its tip to its connection with the catheter body 102 is free from structural obstructions. Stated another way, the inner sloping surface 502 is substantially continuous and thereby without any interruptions (e.g., humps, projections and the like). That is to say, the distal end 118 of the distal member 114 is substantially continuous (e.g., without obstructions) at the distal end and proximal to the distal end. Accordingly, as thrombus 602 is aspirated into the catheter, the thrombus becomes wedged at the distal end of, or within the distal member 114. The funnel shape of the distal member 114 then seals against the thrombus, and the aspiration pressure of aspiration lumen 110 continues vacuuming and collapsing the thrombus 602 into the gradually narrowing widened aspiration orifice distal member 114 to break it up into smaller pieces that can then fit within and be transferred down the aspiration lumen 110.

FIGS. 6B and 6C show further details of a thrombus being aspirated with FIG. 6B showing a side view of the thrombectomy catheter 110 with the thrombus 602 collapsed within the wide mouth distal member 114 and translated toward the proximal catheter end. FIG. 6C shows a side view of the thrombectomy catheter 110 with the thrombus 602 collapsed (or broken up) into the smaller diameter portion of the catheter 110 and translated toward the proximal catheter end for disposal.

The present wide mouth shape reduces any occurrence of fluid diversion around gaps (e.g., leaks) between the thrombus and the aspiration lumen, which reduce the aspiration pressure (e.g., vacuum) incident on thrombus within the widened aspiration orifice distal member 114. That is to say, the unobstructed annular shape of the widened aspiration orifice distal member 114 allows thrombus to seat along the member and substantially prevents the formation of gaps between the thrombus, and projecting features within the distal member 114. Fluid leaks around the thrombus are thereby substantially minimized and the full vacuum of the aspiration lumen 110 is applied to the thrombus.

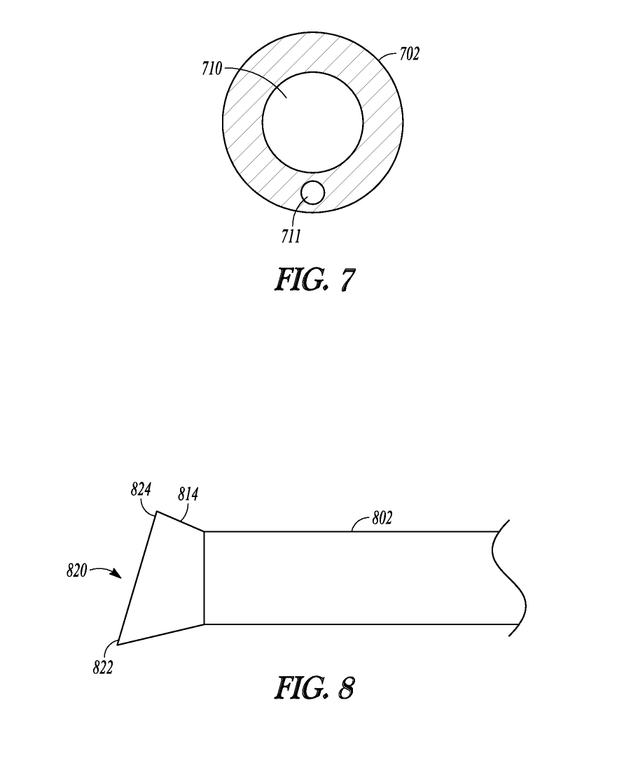

FIG. 7 shows a cross-section of a thrombectomy catheter 702 with the infusion lumen 711 fully positioned with the sidewall of the catheter. The provision of the infusion lumen 711 ensures the profile of the aspiration lumen 710 is substantially isodiametric. In some examples, the isodiametric aspiration lumen 710 (free of obstructions) facilitates the suction and transport of thrombus through the lumen. Alternatively, the catheter 702 includes a portion of the catheter, for instance at the distal or proximal portion 108, 104 that includes the infusion lumen 711 within the sidewall of the catheter while another portion of the catheter, such as the proximal or distal portion 104, 108, includes another part of the infusion lumen 711 partially presented within the aspiration lumen (as shown in FIG. 2) The catheter 702 thereby includes an isodiametric aspiration lumen 710 free of obstructions where needed to efficiently deliver thrombus proximately through the aspiration lumen.

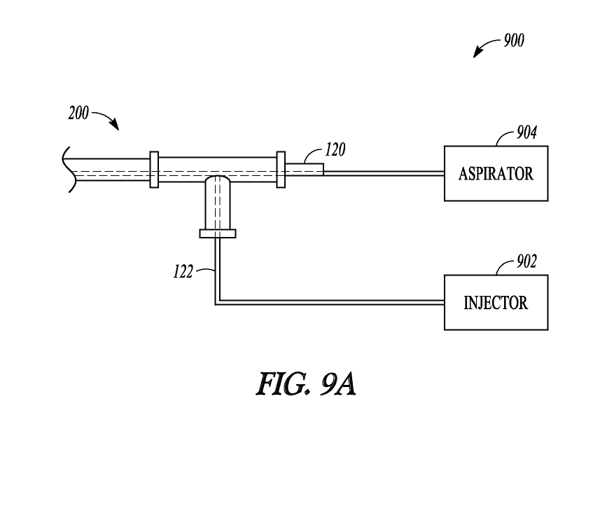

FIG. 8 shows a distal end of a thrombectomy catheter 802, in accordance with another embodiment. In this example, the end of a wide mouth distal member 814 has a beveled shape 815. The beveled shape of the widened aspiration orifice distal member 814 assists with device insertion and navigation into a vessel. The beveled shape 815 tapers from a distal tip 822 slanting up to a proximal portion 824. An aspiration orifice 820 of the widened aspiration orifice distal member 814 accepts thrombus. The beveled shape performs similar to the wide mouth distal member 114 as described above. Accordingly, the widened aspiration orifice distal member 814 is free from any structural obstructions, seats annularly against thrombus within the distal member 814 and forms a seal against any thrombus to prevent fluid diversion around gaps between the thrombus and the aspiration lumen.

### Pressurized Fluid Delivery System

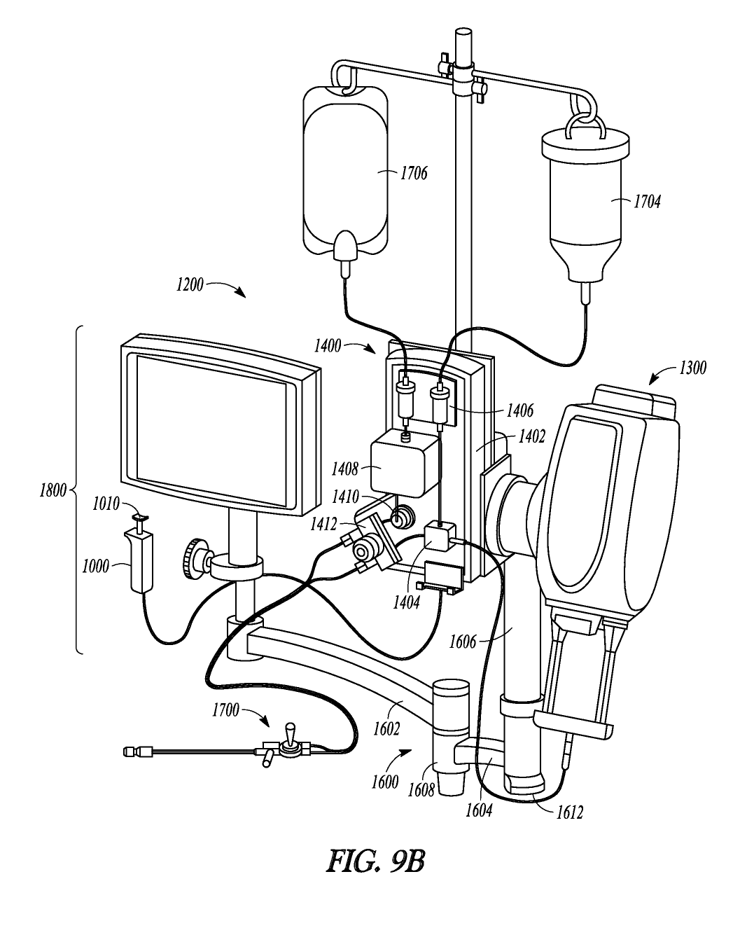

FIG. 9A shows a portion of a thrombectomy system 900, in accordance with one embodiment. The thrombectomy system 900 includes the thrombectomy catheter 100 shown in FIG. 1 with the side port 122 coupled to a fluid delivery device, such as injector 902, and the central port coupled to an aspirator 904, such as a vacuum source.

In use, the thrombectomy catheter 100 is inserted into a vessel using a guidewire, for example. The distal portion 108 of the thrombectomy catheter 100 is navigated through the vasculature placed adjacent a thrombus location. The injector 902 is set to deliver infusion fluid at about 1 cc/s, 1.5 cc/s, 2 cc/s, or 3 cc/s and the like, for example. The injector 902 includes, but is not limited to, a low pressure injector configured for one or more of contrast or medication delivery. A low pressure fluid source is configured to provide infusion fluid to the thrombectomy catheter 100 (802) in a range of between around 300 psi to 2000 psi. As discussed above, the infusion lumen 111 and the infusion orifice 304 are configured by way of shape and diameter to provide a fluid jet having desired flow characteristics (e.g., velocity and flow rate) configured to remove and macerate thrombus according to these lower fluid pressures provided by the injector 902 (as well as lower flow rates compared to high pressure and high flow fluid sources used in other thrombectomy procedures). As discussed herein, the provision of a single infusion orifice 304 localizes the fluid jet to a single location and allows for the use of lower pressure fluids while still removing thrombus. Stated another way, the single infusion orifice 304 avoids the pressure drop across multiple jet orifices, and instead concentrates the hydrodynamic energy provided the low pressure injector 902 at a single location. Other examples can use other fluid delivery devices such as hand-held injectors, high pressure injectors (e.g., 10,000 psi) and the like. The thrombectomy catheter 100 described herein with infusion orifice 304 and infusion lumen 111 provides a thrombectomy system configured to effectively remove and macerate thrombus while using low pressure and low flow rate (e.g., medication and contrast) injectors including continuous delivery pumps without requiring expensive and dedicated high pressure fluid sources (e.g., pumps, injectors and the like).

As the infusion fluid removes and breaks up thrombus, the aspirator 904 coupled with the aspiration lumen 110 is activated to aspirate the particles. The aspirator 904 include a vacuum source, such as a vacuum syringe, vacuum pump and the like.

Another embodiment of an injector system 1200 usable with the present system such as the Medrad Avanta® injector system, is illustrated in FIG. 9B. This example uses a control module 1400, and a powered injector 1300 to which a syringe is connected. The fluid control module 1400 is associated with the injector 1300 for controlling fluid flows delivered by the injector 1300. The fluid control module 1400 is generally adapted to support and control a fluid path set used to connect a syringe associated with the injector 1300 to a catheter (not shown) to be associated with a patient. A source of saline 1706 is in fluid connection with a peristaltic pump 1408.

The fluid delivery system 1200 further includes a support assembly 1600 adapted to support the injector 1300 and the fluid control module 1400, as discussed further herein. The support assembly 1600 may be configured as a movable platform or base so that the fluid delivery system 1200 is generally transportable, or for connection to a standard hospital bed or examination table on which a patient will be located during an injection procedure. Additionally, the fluid delivery system 1200 preferably further includes a user-input control section or device 1800 for interfacing with computer hardware/software (i.e., electronic memory) of the fluid control module 1400 and/or the injector 1300. The fluid control module 1400 generally includes a housing 1402, a valve actuator 1404 for controlling a fluid control valve, a fluid level sensing mechanism 1406, a peristaltic pump 1408, an automatic shut-off or pinch valve 1410, and an air detector assembly 1412.

As indicated, the fluid control module 1400 is generally adapted to support and control the fluid path set 1700 used to connect a syringe associated with the injector 1300 to a catheter (not shown). In a general injection procedure involving the fluid delivery system 1200, the injector 1300 is filled with fluid from the primary fluid container 1704 and delivers the fluid via the fluid path set 1700 to the catheter and, ultimately, the patient. The fluid control module 1400 generally controls or manages the delivery of the injection through a valve associated with the fluid path set 1700, which is controlled or actuated by the valve actuator 1404 on the fluid control module 1400.

The fluid control module 1400 is further adapted to deliver the fluid from the secondary fluid container 1706 under pressure via the peristaltic pump 1408 on the fluid control module 1400. In one embodiment, a handheld controller 1000 includes a plunger or stem control 1010 that, when in a first/low pressure mode, is depressed by the operator to control the flow of fluid from syringe 1300. The farther plunger 1010 is depressed, the greater the flow rate (via, for example, a potentiometer such as a linear potentiometer within the housing of controller 1000). In one embodiment, the operator can use graphical user interface display to change the mode of plunger 1010 to a second mode in which it causes injector 1300 to initiate a high pressure injection as preprogrammed by the operator.

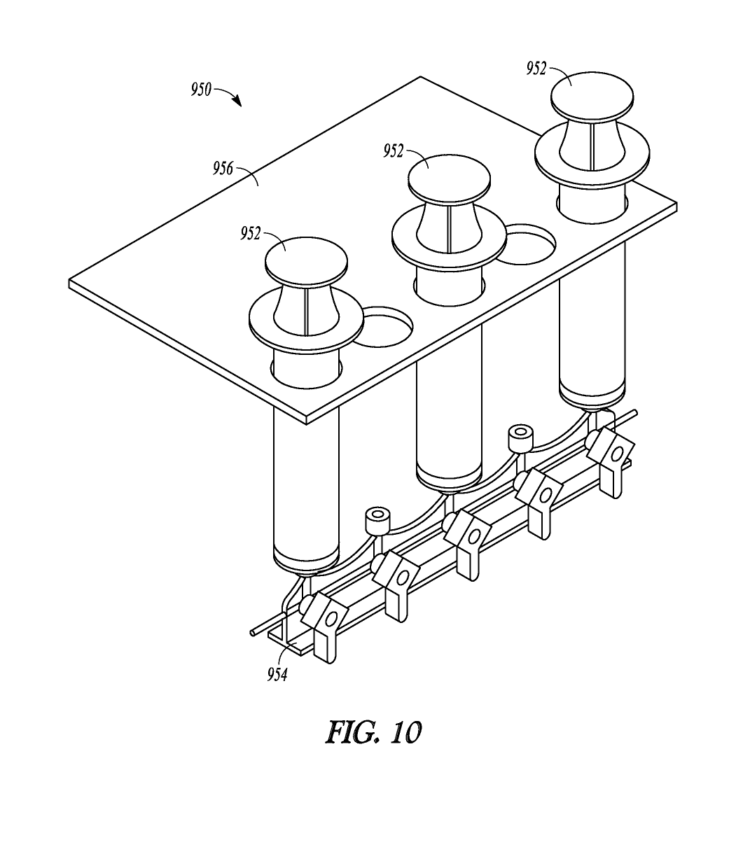

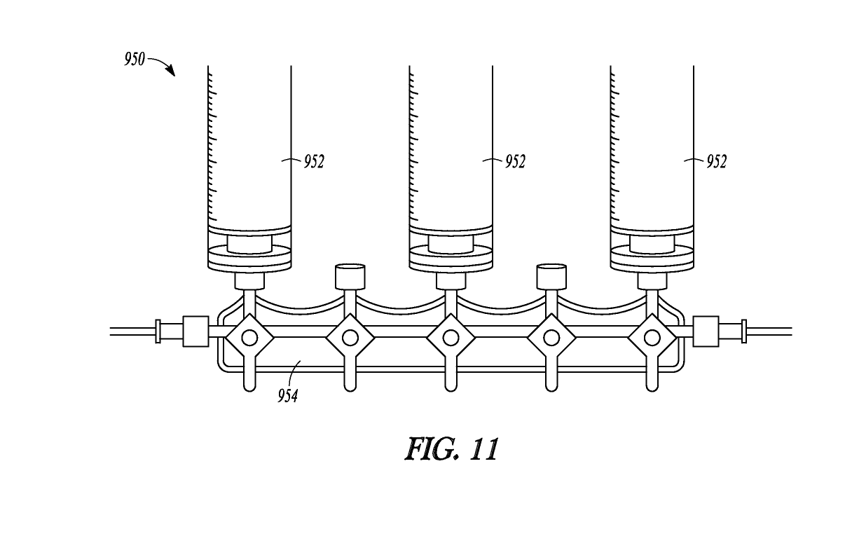

FIG. 10 shows a perspective view of a vacuum source 950, in accordance with one embodiment. FIG. 11 shows a front view of the vacuum source 950.

In this example, the vacuum source 950 is a resettable vacuum source. In one example, the present system described above infuses via a saline filled automated contrast injector with a syringe volume of 150 cc. In the example, an aspiration volume of similar size is used with the aspiration style device (e.g., the vacuum source 950). For example, if a standard 30 cc syringe were used with the injector 902, then the procedure would stop when a corresponding 30 cc syringe of the vacuum source 950 was full to avoid the net subtraction or addition of fluid to the anatomy.

In the example shown in FIGS. 10 and 11 the vacuum source 950 includes a series of 60 cc syringes 952 ganged together via a stop cock style manifold 954. Those of skill in the art would appreciate that varying numbers and sizes of syringes can be used. Optionally, the multiple syringes 952 have more capacity than the infusion source (e.g., the injector 902) and are all resettable prior to any procedure. A frame holder 956 is attached to the vacuum source assembly 950 in one example to keep the syringes 952 upright and visible (and correspondingly hands free). Any number of syringes 952 may be utilized depending on the size of the manifold 954 and the desired aspiration (and injector volume).

In use, the vacuum source 950 (e.g., the aspirator 904) is attached via a luer connector to the thrombectomy catheter 100 and one or more of the stopcocks are opened. After the aspirator 904 is turned on, the aspirated material funneled into the catheter 100, for instance through the widened aspiration orifice distal member 114 and thereafter delivered down the aspiration lumen 110, enters the one or more syringes 952 that have been opened. After one or more of the syringes are filled additional syringes 952 are opened if additional aspiration is needed. If the procedure is complete, the syringes 952 are closed, such as with the stopcock manifold 954, and the syringes 952 are replaced or cleaned as needed for the next procedure.

### Injection Systems

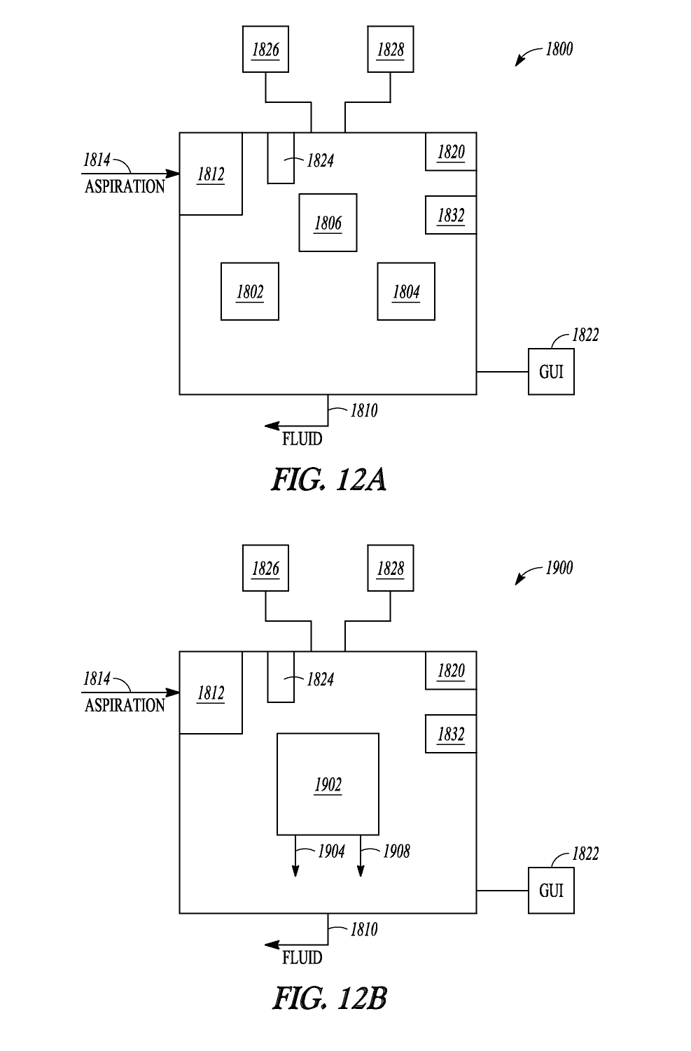

FIG. 12A shows a schematic view of one example of an injector system 1800, according to one embodiment of the present disclosure. FIG. 12B shows a schematic view of another example of an injector system 1900, according to one embodiment of the present disclosure.

These injector systems 1800, 1900 are fluid management mechanisms that can be used with various diagnostic and interventional catheters. The systems incorporate various fluid delivery and management capabilities.

Referring to FIG. 12A, injector system 1800 includes a high-pressure single piston pump 1802. This pump 1802 is configured to provide high-pressure fluid delivery for standard thrombectomy catheters, for example. Some examples provide pressures of about 5,000 psi to about 10,000 psi.

System 1800 further includes a multi-piston pump 1804. Multi-piston pump 1804 is configured to provide medium/low pressure flow for contrast delivery for imaging, flushing agents, and fluid that would be employed using the thrombectomy catheter 100 discussed above. Multi-piston pump 1804 is configured to pump contrast and saline at about 1500 psi and flows of up to 50 ml/sec. Some options have a delivery pressure of about 1000 psi. Some can range from 500 psi to 2500 psi. Pump 1804 is a continuous flow pump (i.e. it does not have to refill like a syringe pump).

One option further includes a single piston pump 1806. Pump 1806 is a pump configured to pump contrast or saline at 1500 psi and flows of up to 50 ml/sec, but it must be refilled. In some embodiments of system 1800, pump 1806 is omitted or pump 1804 is omitted.

Each of pumps 1802, 1804, and 1806 are operatively coupled to an outlet fluid line 1810 to deliver fluid to a catheter or other tool. Pumps 1802, 1804, and 1806 are designed to operate independently, in that only one pump would deliver-fluid at one time.

System 1800 further includes an aspiration module 1812. Aspiration module 1812 is configured to withdraw fluids through either the fluid delivery catheter or a separate catheter.

Each of pumps 1802, 1804, and 1806 are configured to share a common architecture. For example, system 1800 can optionally include operating an power system 1820, a graphical user interface (GUI) 1822, a fluid assurance/air detection module 1824, and one or more bulk fluid sources 1826, 1828. On option includes a module 1832 configured to provide fluid mixing dynamically and monitoring remaining volumes of fluid 1826, 1828. Some options further provide for multi-use disposable, interface and informatics connectivity, and catheter/disposable recognition.

In different embodiments, certain features discussed above are combined in different ways. One example configuration combines pumps 1802 and 1804 with aspiration module 1812, and at least one or more of a standard thrombectomy catheter, a thrombectomy catheter 100 or a diagnostic catheter. Another example configuration combines pumps 1802 and 1806 with the aspiration module 1812, and at least one or more of a standard thrombectomy catheter, thrombectomy catheter 100 or a diagnostic catheter. Still another example configuration combines the pump 1804 with the aspiration module 1812 and one or more of thrombectomy catheter 100 or a diagnostic catheter. An additional configuration combines pump 1806 with aspiration module 1812 and one or more of thrombectomy catheter 100 or a diagnostic catheter. Yet another example configuration combines pump 1806 and one or more of thrombectomy catheter 100 or a diagnostic catheter. Another configuration includes a single pump piston 1806 and is capable of working with a contrast injector or with the thrombectomy catheter 100 discussed above. Conversely, the first described configuration has more complexity because it is compatible with all catheters and capabilities.

By providing all the different capabilities in one compact system, fluid injection system 1800 can be used for multiple cases. Typical injection systems are either high-pressure or low-pressure and so a medical staff must have both systems and be capable of using both. By combing the systems, injector system 1800 is more likely to be used as the set-up is minimal and the learning curve is reduced. Additional benefits include time savings, reduced consumables, additional floor space and availability of a device for any procedure.

Referring to FIG. 12B, injector system 1900 can include any of the features discussed above for injector system 1800, and those features will not be discussed. Similarly, the same multiple use configurations utilizing various catheters can also be utilized.

Here, injector system 1900 includes a multi-piston pump 1902 that is capable of delivering low pressure fluids 1904 for use in contrast imaging, flushing solutions, or use with thrombectomy catheter 100 discussed above. Further multi-piston pump 1904 can deliver high pressure fluids 1908 for use with standard thrombectomy catheters.

Again, by providing all the different capabilities in one compact system, fluid injection system 1900 can be used for multiple cases. Typical injection systems are either high-pressure or low-pressure and so a medical staff must have both systems and be capable of using both. By combing the systems, injector system 1900 is more likely to be used as the set-up is minimal and the learning curve is reduced. Additional benefits include time savings, reduced consumables, additional floor space and availability of a device for any procedure.

### Thrombectomy Catheter

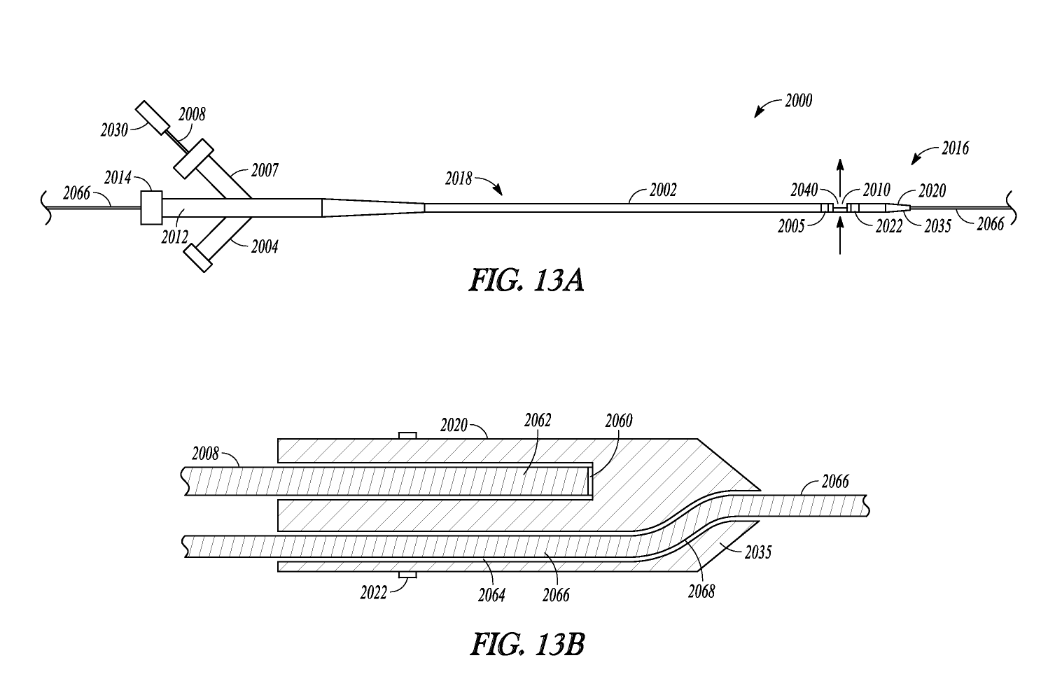

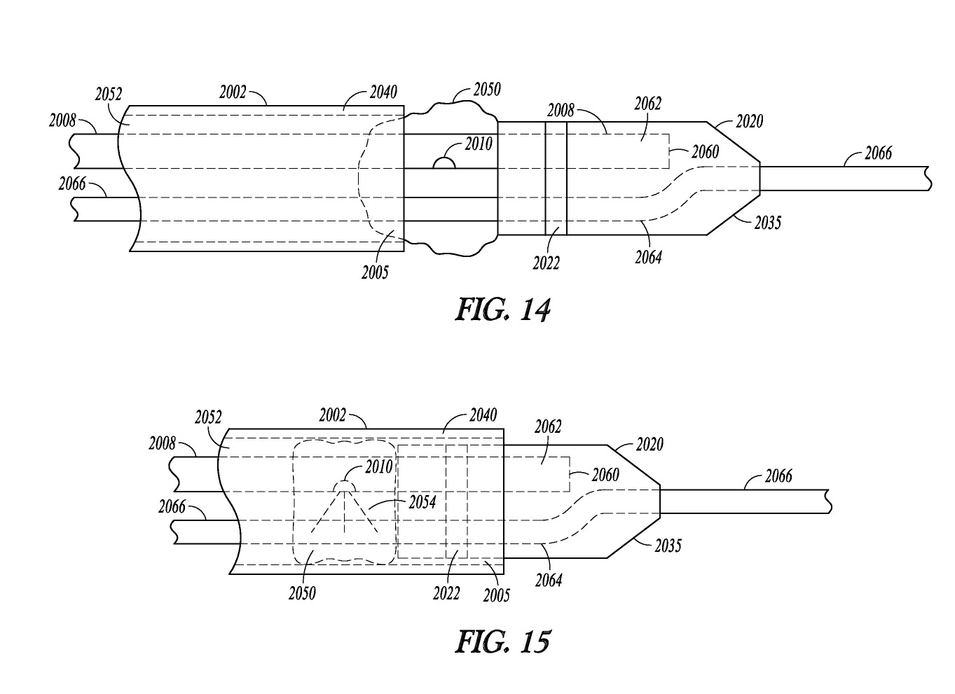

FIG. 13A shows a side view of an embodiment of a thrombectomy catheter 2000 according to one or more embodiments of the present disclosure. Thrombectomy catheter 2000 generally includes a catheter body 2002 which includes an aspiration lumen 2052 (see FIGS. 14 and 15) extending though the catheter body 2002 and open at a distal end at an aspiration orifice 2005. The aspiration lumen 2052 communicates with an aspiration port 2004 which can be coupled to a vacuum source as discussed above, for instance with a hemostasis valve, fitting or the like. The vacuum source includes, but is not limited to, a syringe, vacuum bottle, roller pump, vacuum pump or the like. The thrombectomy catheter 2000 includes a fluid injection port 2007 (similarly including a hemostatis valve, fitting or the like). In this example, fluid can be delivered through an infusion body 2008, such as a stainless steel hypotube, polymer tube, Nitinol tube or the like. Infusion body 2008 can include a connection member 2030 for connection to an injector source, such as the injectors discussed above. Infusion body includes an internal lumen extending through the infusion body and having an infusion orifice 2010 near a catheter distal portion 2016. In one example, a single infusion orifice 2010 is used. Infusion body extends through the catheter body 2002 within the aspiration lumen 2052.

FIG. 13A further shows a guide wire 2066 extending through the catheter body 2002 and an expanded member (2020, described below). The guide wire 2066 facilitates navigation through the vasculature and further allows for sliding movement of the components of the thrombectomy catheter relative to one another while maintaining coincidence of the infusion body 2008 (and the expanded member) relative to the catheter body 2002. As shown in FIG. 13A, the guide wire 2066 extends through a manifold 2012 coupled with a catheter proximal portion 2018. The guide wire 2066 enters the manifold 2012 through an access port 2014. As with the other ports, including the aspiration port 2004 and the fluid injection port 2007, a hemostasis valve is optionally provided at the access port 2014 to facilitate the sealed delivery of the guide wire 2066 through the manifold 2012. The sealed environment provided within the thrombectomy catheter 2000 allows for aspiration of infusion fluids including entrained particulate from the distal end of the infusion body 2008 and the catheter distal portion 2016 (e.g., adjacent to the expanded member 2020 at the aspiration orifice 2005).

Coupled to a distal end of the infusion body 2008 and located distally from the infusion orifice 2010 is an expanded member 2020. The expanded member 2020 is shown in FIG. 13B as a detailed cross section. The expanded member 2020 includes a diameter that is larger than the infusion body 2008. In one example expanded member 2020 is dimensioned to fit within the aspiration orifice 2005. In one example, expanded member 2020 includes a tapered distal portion 2035 and one or more marker bands 2022. In some examples, the expanded member 2020 includes a glue bulb or an additional coil of wire. In other examples, the aspiration lumen 2052 includes a widened aspiration orifice distal member 114, for instance as shown in FIGS. 3 and 4. Optionally, the expanded member 2020 is tapered near a proximal end to facilitate delivery into the aspiration orifice 2005.

As further shown in FIG. 13B, the expanded member 2020 includes an infusion body recess 2060 sized and shaped to receive an infusion body distal end 2062 (e.g., the distal end of a hypotube providing the infusion fluid to the fluid infusion orifice 2010). In one example the infusion body distal end 2062 is fixedly coupled with the expanded member with at least one mechanism including, but not limited to, adhesives within the infusion body recess 2060, crimping, overmolding, mechanical interference fitting and the like. In another example, the expanded member 2020 is sized and shaped for sliding reception of an instrument, such as a guide wire within a guide wire passage 2064. In FIG. 13B, the guide wire 2066 is shown extending through the expanded member 2020. Optionally, the guide wire passage 2064 includes a passage that is at least partially non-linear as shown, including for instance an elbow 2068. In another option, the guide wire passage 2064 is substantially centrally located within the expanded member 2020. The expanded member 2020 rides over the guide wire 2066 with the guide wire 2066 acting as a rail. In yet another option, the guide wire passage is provided within the infusion body 2008 and accordingly consolidates the guide wire and the infusion body 2008 in a coincident configuration.

The expanded member 2020 is configured to free plugs of material 2050 that are lodged within the aspiration orifice 2005. For example, plugs 2050 of thrombus plug the tip 2040 of the thrombectomy catheter 2000, as shown in FIG. 14. Retraction of the infusion body 2008 (or conversely translational advancement of the catheter tip 2040 past the expanded member 2020) as shown in FIG. 15 frees the blockage, thus restoring aspiration without the need to remove the catheter from the body. Optionally, the expanded member 2020 and the infusion body 2008 are removable from the catheter body 2002, for instance by proximal or distal sliding of the infusion body 2008 relative to the catheter body. In yet another example, the expanded member 2020 and the infusion body 2008 are provided as a unitary device sized shaped for use with one or more standard delivery or interventional catheters having interior lumens sized to receive the infusion body 2008 and the expanded member 2020 therein.

In operation, thrombus plugs the aspiration orifice 2005. The user manipulates the infusion body 2008 by one or more of rotating the infusion body 2008 in either direction (clockwise or counterclockwise) and by reciprocating the infusion body longitudinally relative to the catheter body 2002. As shown in FIGS. 14 and 15 the expandable member 2020 translates as a slidable element relative to the guide wire 2066 received within the guide wire passage 2064. The guide wire 2066 according serves as a rail for the expanded member 2020. The guide wire 2066, also received in the aspiration lumen 2052, assists in centering the expandable member 2020 relative to the aspiration lumen 2052.

When the expanded member 2020 is within the aspiration lumen 2052 it physically pushes (e.g., plunges, mechanically engages and the like) the thrombus 2050 into and down the aspiration lumen 2052. At the same time, the infusion orifice 2010 is positioned inside the aspiration lumen 2052 of the catheter body 2002 and the infusion jet 2054 assists in breaking up the thrombus 2050. The expanded member 2020 acts as a plug for the aspiration orifice 2005 and the infusion jet will be directed toward the blocking thrombus, and the outflow of the infusion jet 2054 from the infusion orifice 2010 will carry the thrombus through the aspiration lumen 2052. Stated another way, the infusion orifice 2010 and the generated infusion jet 2054 cooperate with the mechanical engagement (e.g., plunging) provided by the expanded member to dislodge plugs 2050 of material at the aspiration orifice 2005 and within the aspiration lumen 2052. This combined functionality minimizes and substantially eliminates plugging of the aspiration lumen 2052 even with the delivery of low pressure infusion fluids through the infusion orifice 2010.

In the present example, the expanded member 2020 cooperates with the catheter body 2002 to remove thrombus 2050 in such a manner that the present example can eliminate the wider aspiration orifice distal member 114, discussed above. This allows the device to smoothly track through blockages and vasculature without embolization or vessel damage. In some embodiments, the wider aspiration orifice distal member 114 can be used with the expanded member 2020.

### Additional Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

We claim:

1. A thrombectomy catheter comprising:
a catheter body extending from a catheter proximal portion to a catheter distal portion and including a catheter intermediate portion, the catheter body including an aspiration lumen and an infusion lumen extending along the catheter body, the catheter body having an integral homogenous cross-section profile and having a multi-durometer hardness varying along the catheter body's length such that the catheter proximal portion has a relatively high durometer value and the catheter distal portion has a relatively low durometer value, with respect to each other;
wherein the aspiration lumen includes an aspiration orifice open at a distal end of the catheter body; and
wherein the infusion lumen extends along the catheter body towards the distal portion and includes a single infusion orifice that is configured to direct a fluid jet radially away from a longitudinal axis of the catheter body.

2. The thrombectomy catheter of claim 1, wherein the distal end of the catheter body includes an aspiration orifice distal member including a proximal portion extending from the distal end of the catheter body having an opening sized similar to the aspiration lumen and a distal portion having an opening wider than the aspiration lumen.

3. The thrombectomy catheter of claim 1, wherein the aspiration lumen is free from structural obstructions along a length of the catheter body.

4. The thrombectomy catheter of claim 1, wherein the aspirating orifice includes a circular cross-section shape.

5. The thrombectomy catheter of claim 1, wherein the single infusion orifice is recessed proximally away from the aspiration orifice.

6. A thrombectomy catheter comprising:
a catheter body extending from a catheter proximal portion to a catheter distal portion;
an aspiration lumen extending through the catheter body from the catheter proximal portion toward the catheter distal portion, the aspiration lumen including an aspiration orifice near the catheter distal portion, wherein the distal end of the catheter body includes an aspiration orifice distal member including a proximal portion extending from the distal end of the catheter body having an opening sized similar to the aspiration lumen and a distal portion having an opening wider than the aspiration lumen; and
an infusion lumen extending along the catheter body towards the distal portion and having a single infusion orifice located in a side wall of the catheter body that is configured to direct a fluid jet radially away from a longitudinal axis of the catheter body.

7. A thrombectomy catheter comprising:
a catheter body extending from a catheter proximal portion to a catheter distal portion and including a catheter intermediate portion, wherein the catheter proximal portion has a relatively high durometer value and the catheter distal portion has a relatively low durometer value, with respect to each other;
the catheter body including an aspiration lumen extending through the catheter body from the catheter proximal portion toward the catheter distal portion, the aspiration lumen including an aspiration orifice open at a distal end of the catheter body, wherein the aspirating orifice is free from structural obstructions at the distal end of the catheter body and wherein the distal end of the catheter body includes an aspiration orifice distal member including a proximal portion extending from the distal end of the catheter body having an opening sized similar to the aspiration lumen and a distal portion having an opening wider than the aspiration lumen;
the catheter body further including an infusion lumen extending along the catheter body towards the distal portion with an infusion orifice extending through the catheter body to direct a fluid jet away from the catheter body.

8. The thrombectomy catheter of claim 7, wherein the infusion orifice includes a single infusion orifice that is configured to direct a fluid jet radially away from a longitudinal axis of the catheter body.

9. A thrombectomy catheter comprising:
a catheter body extending from a catheter proximal portion to a catheter distal portion and including a catheter intermediate portion, the catheter body including an aspiration lumen and an infusion lumen extending along the catheter body, the catheter body having an integral homogenous cross-section profile and having a multi-durometer hardness varying along the catheter body's length such that the catheter proximal portion has a relatively high durometer value and the catheter distal portion has a relatively low durometer value, with respect to each other;
wherein the aspiration lumen extends through the catheter body from the catheter proximal portion toward the catheter distal portion, the aspiration lumen including an aspiration orifice open at a distal end of the catheter body, wherein the distal end of the catheter body includes an aspiration orifice distal member including a proximal portion extending from the distal end of the catheter body a distal portion, wherein the distal portion has a greater cross-sectional area than the cross-sectional area of the proximal portion; and
wherein the infusion lumen extends along the catheter body towards the distal portion with an infusion orifice extending through the catheter body to direct a fluid jet away from the catheter body.

10. A thrombectomy catheter comprising:
a catheter body extending from a catheter proximal portion to a catheter distal portion and including a catheter intermediate portion, the catheter body including an aspiration lumen and an infusion lumen extending along the catheter body, the catheter body having an integral homogenous cross-section profile and having a multi-durometer hardness varying along the catheter body's length such that the catheter proximal portion has a relatively high durometer value and the catheter distal portion has a relatively low durometer value, with respect to each other;
wherein the aspiration lumen extends through the catheter body from the catheter proximal portion toward the catheter distal portion, the aspiration lumen including an aspiration orifice open at a distal end of the catheter body; and
wherein the infusion lumen extends along the catheter body towards the distal portion with an infusion orifice extending through the catheter body to direct a fluid jet away from the catheter body.

11. A thrombectomy system comprising:
a fluid delivery device;
an aspirator; and
a thrombectomy catheter with a first port coupled to the fluid delivery device and a second port coupled to the aspirator, wherein the thrombectomy catheter includes:
a catheter body extending from a catheter proximal portion to a catheter distal portion and including a catheter intermediate portion, wherein the catheter proximal portion has a relatively high durometer value and the catheter distal portion has a relatively low durometer value, with respect to other;
an aspiration lumen extending through the catheter body from the catheter proximal portion toward the catheter distal portion, the aspiration lumen including an aspiration orifice open at a distal end of the catheter body, wherein the distal end of the catheter body includes an aspiration orifice distal member including a proximal portion extending from the distal end of the catheter body having an opening sized similar to the aspiration lumen and a distal portion having an opening wider than the aspiration lumen;
the catheter body further including an infusion lumen extending along the catheter body towards the distal portion with an infusion orifice extending through the catheter body to direct a fluid jet away from the catheter body.

12. The thrombectomy system of claim 11, wherein the aspirator includes a vacuum source including a plurality of syringes ganged together via a stop cock style manifold.

13. A thrombectomy catheter comprising:
a catheter body including an aspiration lumen extending though the catheter body and open at an aspiration orifice;
an infusion body including a fluid delivery lumen extending to an infusion orifice, the infusion body extending through the aspiration lumen; and
an expanded member coupled to a distal end of the infusion body and located distally from the infusion orifice.

14. The thrombectomy catheter of claim 13, wherein the expanded member includes a size that is larger than a cross-section size of the infusion body.

15. The thrombectomy catheter of claim 13, wherein the expanded member is dimensioned to fit at least partially within the aspiration orifice.

16. The thrombectomy catheter of claim 13, wherein the infusion body includes a single infusion orifice.

17. The thrombectomy catheter of claim 13 wherein the thrombectomy catheter is configured such that the expanded member can be pulled back within the aspiration lumen to contact any thrombus lodged within the aspiration orifice.

18. A method comprising:
performing a thrombectomy procedure with a thrombectomy catheter having an aspiration lumen and an infusion body extending through the aspiration lumen, the infusion body including an infusion orifice and an expanded member located distally from the infusion orifice;
manipulating the infusion body to move the expanded member into the aspiration lumen, the expanded member is engaged with a thrombus blockage; and moving the thrombus blockage into the aspiration lumen with the expanded member.

19. The method of claim 18, further comprising locating the infusion orifice within the aspiration lumen to hydrodynamically macerate the thrombus blockage.

20. The method of claim 18, comprising infusing infusion fluid into the aspiration lumen, wherein the aspiration lumen is closed with one or more of the expanded member or the thrombus blockage at an aspiration orifice.

21. The method of claim 18, wherein manipulating the infusion body includes manipulating the infusion body to move the expanded member into the aspiration lumen with the thrombus blockage initially interposed between an aspiration orifice of the catheter body and the expanded member.

22. An injector system comprising:
a housing holding a high pressure pump, a low pressure pump, and an aspiration module;
wherein a thrombectomy catheter is configured for coupling to the high pressure pump or the low pressure pump;
the high pressure pump further comprising a single piston pump capable of delivering fluid at pressures of between 5000 psi to 10,000 psi; and
the low pressure pump further comprising a multi-piston pump capable of delivering fluids at between 500 psi to 1500 psi.

23. The injector system of claim 22, further including a single piston pump configured to pump fluid at about 1500 psi.

24. The injector system of claim 22, wherein the high pressure pump and the low pressure pump are configured to operate independently of each other.
